(19) United States
(12) Patent Application Publication
(10) Pub. No.: US 2026/0207375 A1
(43) Pub. Date: Jul. 23, 2026

(54) UTERINE HEAT PATCH

(71) Applicant: Fridababy, LLC, Miami, FL (US)

(72) Inventors: Janette VILLALOBOS, Miami Springs, FL (US); Sophie FORNARO, Miami, FL (US); Chelsea HIRSCHHORN, Miami Beach, FL (US)

(21) Appl. No.: 19/455,508

(22) Filed: Jan. 21, 2026

Related U.S. Application Data
(60) Provisional application No. 63/748,334, filed on Jan. 22, 2025.

(51) Int. Cl. A61F 7/02; C08J 5/22; C08L 23/06; A61F 7/00

(52) U.S. Cl. CPC A61F 7/02; C08J 5/2231; C08L 23/06; A61F 2007/0022; A61F 2007/0226

(57) ABSTRACT

A heat patch may comprise a heat-generating component configured to produce sustained heat through an exothermic chemical reaction. The heat-generating component may comprise an iron-based composition. A non-woven topsheet may form a skin-facing surface. The topsheet may be breathable. A first polyethylene film layer may be positioned between the topsheet and the heat-generating component. A non-woven backing layer may comprise an adhesive for attaching the heat patch to the inside of clothing. A release layer may cover the adhesive. The heat patch may be contoured to fit an abdominal region. The heat-generating component may be activated by exposing the heat-generating component to air. The heat patch may be attached to an inner surface of clothing positioned over a postpartum abdominal region. The heat patch may deliver heat to the postpartum abdominal region for a sustained period.

### CROSS-REFERENCE TO RELATED APPLICATION

The present application claims priority to U.S. Provisional Application No. 63/748,334, filed on January 22, 2025, and titled “UTERINE HEAT PATCH,” the disclosure of which is expressly incorporated by reference in its entirety.

### BACKGROUND

### Field

Aspects of the present disclosure generally relate to a heat patch, and more specifically to a uterine heat patch.

### Background

Heat patches may be used as therapeutic devices to provide localized warmth for pain relief and muscle relaxation. These patches typically function by generating heat through an exothermic chemical reaction, often involving iron powder, activated carbon, and other ingredients that oxidize when exposed to air. The heat generated by such reactions is steady and can be sustained over several hours, making heat patches desirable for temporary relief from various types of discomfort.

### SUMMARY

In some aspects, a heat patch may include a heat-generating component configured to produce sustained heat through an exothermic chemical reaction. The heat-generating component may comprise an iron-based composition. A non-woven topsheet may form a skin-facing surface. The topsheet may be breathable. A first polyethylene (PE) film layer may be positioned between the topsheet and the heat-generating component. A non-woven backing layer may comprise an adhesive for attaching the heat patch to the inside of clothing. A release layer may cover the adhesive. The heat patch may be contoured to fit an abdominal region.

In some aspects, a method of manufacturing a heat patch may include laminating a first polyethylene (PE) film layer to a non-woven topsheet such that the first PE film layer is positioned between a non-woven topsheet and a heat-generating component. The heat-generating component may comprise an iron-based composition. The method may comprise disposing the heat-generating component adjacent to the first PE film layer on a side of the first PE film layer opposite the non-woven topsheet. The method may comprise joining a non-woven backing layer to the heat-generating component such that the adhesive is positioned on a side of the non-woven backing layer opposite the heat-generating component. The non-woven backing layer may comprise an adhesive configured for attaching the heat patch to an inside surface of an article of clothing. The method may comprise cutting the laminated structure to define a contoured shape configured to fit an abdominal region. The heat patch may comprise a group of segmented panels. Each panel of the group of segmented panels may include a pouch. Each pouch may include the heat-generating component.

In some aspects, a method for using a heat patch may include activating a heat-generating component within the heat patch by exposing the heat-generating component to air. The heat-generating component may comprise an iron-based exothermic formulation. The method may comprise attaching the heat patch to an inner surface of clothing positioned over a postpartum abdominal region, such that the heat patch delivers heat to the postpartum abdominal region for a sustained period. The heat patch may comprise a non-woven topsheet forming a skin-facing surface. The topsheet may be breathable. The heat patch may comprise a first polyethylene (PE) film layer positioned between the non-woven topsheet and the heat-generating component. The heat patch may comprise a non-woven backing layer comprising an adhesive for attaching the heat patch to the inside of clothing. The heat patch may comprise a release layer covering the adhesive prior to application. The method may further comprise positioning the heat patch to align with the postpartum uterine area to deliver the heat. The heat patch may be attached to postpartum underwear using an adhesive layer on the backing of the heat patch. The method may further comprise applying the heat patch to alleviate afterpains caused by oxytocin-induced uterine contractions. The heat patch may further comprise a second PE film layer positioned between the heat-generating component and the non-woven backing layer.

In some aspects, a method for heat therapy may include providing heat therapy via a heat-generating component within a heat patch, the heat-generating component comprising an iron-based exothermic formulation. The heat patch may include a non-woven topsheet forming a skin-facing surface, the topsheet being breathable. The heat patch may also include a first polyethylene (PE) film layer positioned between the non-woven topsheet and the heat-generating component. The heat patch may further include a non-woven backing layer comprising an adhesive layer for attaching the heat patch to the inside of clothing. The heat patch may also include a release layer covering the adhesive prior to application.

Aspects generally include a method and a device as substantially described with reference to and as illustrated by the accompanying drawings and specification.

The foregoing has outlined rather broadly the features and technical advantages of examples according to the disclosure in order that the detailed description that follows may be better understood. Additional features and advantages will be described. The conception and specific examples disclosed may be readily utilized as a basis for modifying or designing other structures for carrying out the same purposes of the present disclosure. Such equivalent constructions do not depart from the scope of the appended claims. Characteristics of the concepts disclosed, both their organization and method of operation, together with associated advantages will be better understood from the following description when considered in connection with the accompanying figures. Each of the figures is provided for the purposes of illustration and description, and not as a definition of the limits of the claims.

### BRIEF DESCRIPTION OF THE DRAWINGS

So that features of the present disclosure can be understood in detail, a particular description may be had by reference to aspects, some of which are illustrated in the appended drawings. It is to be noted, however, that the appended drawings illustrate only certain aspects of this disclosure and are therefore not to be considered limiting of its scope, for the description may admit to other equally effective aspects. The same reference numbers in different drawings may identify the same or similar elements.

FIG. 1 is a diagram illustrating an example of a heat patch, in accordance with various aspects of the present disclosure.

FIG. 2A is a diagram illustrating an example of a heat patch, in accordance with various aspects of the present disclosure.

FIG. 2B illustrates the heat patch in a folded configuration, in accordance with various aspects of the present disclosure.

FIG. 3A is a diagram illustrating an example of a heat patch, in accordance with various aspects of the present disclosure.

FIG. 3B illustrates an example of the heat patch in a folded configuration, in accordance with various aspects of the present disclosure.

FIG. 4 is a diagram illustrating an example of a heat patch, in accordance with various aspects of the present disclosure.

FIG. 5 is a diagram illustrating an example of a heat patch, in accordance with various aspects of the present disclosure.

FIG. 6 is a diagram illustrating an example of a heat patch, in accordance with various aspects of the present disclosure.

FIG. 7 is a flow diagram illustrating an example of a process for using a heat patch in accordance with various aspects of the present disclosure.

FIG. 8 is a flow diagram illustrating an example of a process for manufacturing a heat patch, in accordance with various aspects of the present disclosure.

FIG. 9 illustrates an example process for heat therapy, in accordance with various aspects of the present disclosure.

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. It will be apparent to those skilled in the art, however, that these concepts may be practiced without these specific details. In some instances, well-known structures and components are shown in block diagram form in order to avoid obscuring such concepts.

Based on the teachings, one skilled in the art should appreciate that the scope of the present disclosure is intended to cover any aspect of the present disclosure, whether implemented independently of or combined with any other aspect of the present disclosure. For example, an apparatus may be implemented, or a method may be practiced using any number of the aspects set forth. In addition, the scope of the present disclosure is intended to cover such an apparatus or method practiced using other structure, functionality, or structure and functionality in addition to, or other than the various aspects of the present disclosure set forth. It should be understood that any aspect of the present disclosure may be embodied by one or more elements of a claim.

The word “exemplary” is used herein to mean “serving as an example, instance, or illustration.” Any aspect described herein as “exemplary” is not necessarily to be construed as preferred or advantageous over other aspects.

Although particular aspects are described herein, many variations and permutations of these aspects fall within the scope of the present disclosure. Although some benefits and advantages of the preferred aspects are mentioned, the scope of the present disclosure is not intended to be limited to particular benefits, uses, or objectives. Rather, aspects of the present disclosure are intended to be broadly applicable to different configurations, some of which are illustrated by way of example in the figures and in the following description of the preferred aspects. The detailed description and drawings are merely illustrative of the present disclosure rather than limiting, the scope of the present disclosure being defined by the appended claims and equivalents thereof.

As discussed, heat patches may be used as therapeutic devices to provide localized warmth for pain relief and muscle relaxation. These patches typically function by generating heat through an exothermic chemical reaction, often involving iron powder, activated carbon, and other ingredients that oxidize when exposed to air. The heat generated by such reactions is steady and can be sustained over several hours, making heat patches a convenient and effective solution for temporary relief from various types of discomfort.

Heat therapy may be used to alleviate pain, improve blood circulation, and relax muscles, among other uses. As a result, heat patches are commonly used for conditions such as menstrual cramps, muscle soreness, and joint pain, with product variations tailored to specific applications. Conventional heat patches are lightweight, portable, and easy to use, with self-adhesive backings that allow them to adhere securely to the skin or clothing. Advancements in heat patch technology have led to the development of multi-layered designs that provide both functionality and comfort. These designs often incorporate breathable and soft materials to ensure compatibility with sensitive skin, along with temperature-regulating mechanisms to maintain consistent and safe heating levels.

In some cases, heat therapy may be used for postpartum recovery. Postpartum recovery is a critical period during which new mothers experience significant physical and emotional changes following childbirth. One of the most common challenges during this time is managing uterine pain and discomfort, often referred to as postpartum cramps or afterpains. These cramps are caused by intense uterine contractions as the uterus returns to its pre-pregnancy size, a process known as uterine involution. The pain can be exacerbated by breastfeeding, which stimulates the release of oxytocin and triggers additional contractions.

Despite the widespread prevalence of postpartum cramps, with over one-third of new mothers experiencing moderate to severe discomfort, there is a lack of products specifically designed to address postpartum uterine pain relief. Existing solutions, such as over-the-counter pain medications or generic warming products intended for menstrual cramps, fail to address the unique physiological needs of postpartum recovery. Unlike menstrual cramps, postpartum uterine involution involves the healing of a large internal wound caused by the detachment of the placenta. This process, which takes several weeks, requires targeted care to support uterine contraction, promote blood flow, and accelerate the expulsion of lochia (postpartum discharge).

Additionally, postpartum skin is often more sensitive and prone to irritation due to hormonal changes and the stretching and recovery of the abdominal area. Conventional heat patches designed for general pain relief may not be suitable for postpartum use, as they are not tailored to the contours of a postpartum belly or compatible with the delicate condition of postpartum skin. This unmet need highlights the necessity for a product specifically designed to provide gentle, targeted, and effective uterine pain relief during postpartum recovery.

Conventional heat patches may rely on pressure-sensitive adhesives formulated for room-temperature use and direct skin contact. However, when such adhesives are exposed to sustained elevated temperatures generated by exothermic heat patches, their adhesive properties may change in ways that are undesirable for postpartum use. For example, increased temperature may soften or flow the adhesive, resulting in excessive bonding to delicate textile substrates such as disposable postpartum underwear, mesh garments, or absorbent materials. Upon removal, such excessive adhesion can cause tearing, deformation, or damage to the garment, while insufficient adhesion may cause premature detachment during use. These competing effects present a technical challenge in designing a heat patch suitable for postpartum recovery, particularly when the heat patch is intended to be worn for extended durations.

In some examples, peel strength may be characterized as a normalized measure of adhesive performance and may be expressed, for example, in N/mm. In certain implementations, the peel strength may be within a predetermined range that provides sufficient adhesion to maintain secure attachment during use, while reducing or preventing tearing, deformation, or damage upon removal. By way of non-limiting example, full-adhesive backing uterine heat patches produced using a bulk production adhesive coating of about 25 g/m² may exhibit peel strengths on the order of about 0.05–0.07 N/mm, while patches produced using a bulk production adhesive coating of about 35 g/m² may exhibit peel strengths on the order of about 0.08–0.12 N/mm. In further examples, strip-back adhesive configurations produced using a bulk production adhesive coating of about 47 g/m² may exhibit peel strengths on the order of about 0.12–0.17 N/mm.

In some examples, the uterine heat patch may be configured such that an acceptance criterion for peel strength is less than about 0.10 N/mm on one or more postpartum garment substrates, thereby defining an upper bound that mitigates garment tearing while preserving functional adhesion over extended wear durations and elevated operating temperatures. Other peel strength ranges, upper limits, or substrate-dependent thresholds may be selected to balance adhesion reliability and removability across a useful range of garments, materials, and use conditions, without requiring complete elimination of adhesion to textile substrates.

In addition to adhesive considerations, conventional heat patches often use relatively thin, low-loft outer layers that allow heat to be transferred directly and rapidly to the skin. While this may be acceptable for general muscle or joint pain, such configurations can produce a harsh or localized thermal sensation when applied to postpartum skin, which may be stretched, inflamed, or otherwise sensitive following childbirth or surgical intervention. In contrast, postpartum users may benefit from a heat delivery profile that moderates abrupt thermal sensation while still providing sustained therapeutic warmth. Existing products do not adequately address this balance between effective heat delivery and tactile comfort in the context of postpartum recovery.

Further, many existing heat therapy products are designed primarily for direct application to the skin and do not account for performance when worn in conjunction with garments. In postpartum settings, users may prefer or require heat patches to be worn on or through clothing layers for hygiene, comfort, or convenience. Designing a heat patch that maintains effective heat transfer, reliable adhesion, and garment integrity under these conditions presents additional challenges not addressed by conventional heat patch designs.

In some examples, the heat patch may include different loft materials or thicknesses on opposing sides of the heating element so that the patch can be used in at least two configurations. In a first configuration, the patch is worn in conjunction with a disposable undergarment, where a relatively thinner loft layer is positioned between the heating element and the wearer to increase perceived heat through the undergarment. In a second configuration, a relatively thicker loft layer is positioned toward the wearer when the patch is worn without such an undergarment, while still providing the comfort, thermal regulation, and other benefits described herein.

Various aspects of the present disclosure are directed to a heat patch specifically engineered to address the physiological, mechanical, and comfort-related constraints associated with postpartum uterine recovery. In some examples, the heat patch may be a single-use heat patch. In contrast to conventional heat patches that are designed for general muscle or joint pain, the disclosed heat patch delivers controlled, sustained therapeutic warmth to the postpartum abdominal region while maintaining compatibility with sensitive, recovering skin and delicate garment substrates. In some examples, the heat patch includes a multi-layered construction comprising a heat-generating component activated by an exothermic chemical reaction, one or more intermediate containment and distribution layers, and a skin-facing topsheet selected to moderate thermal sensation and improve tactile comfort during extended wear. The heat patch may be contoured to conform to the postpartum belly, thereby promoting even heat distribution across the uterine region while reducing localized pressure or folding during movement.

In contrast to conventional heat therapy products that rely on adhesives optimized for room-temperature or skin-only application, particular aspects of the present disclosure incorporate attachment mechanisms configured to maintain reliable adhesion to garments under sustained elevated temperatures generated by the heat-generating component. These attachment mechanisms balance competing performance requirements, including maintaining secure placement during use while avoiding excessive bonding, tearing, or deformation of disposable postpartum underwear or similar textile substrates upon removal. In this manner, the disclosed heat patch addresses challenges unique to heated garment-based applications that are not encountered in cooling products or general-purpose heat patches.

In some aspects, the heat patch includes a lofted, non-woven topsheet having a thickness, compressibility, or softness selected to buffer abrupt thermal sensation and reduce harsh localized heat perception at the skin interface. This configuration may be advantageous for postpartum users, whose abdominal skin may be stretched, inflamed, or otherwise aggravated due to childbirth or surgical intervention. By moderating the initial and sustained thermal feel while preserving effective heat transfer, the disclosed heat patch improves user comfort in ways not achieved by thin or low-loft coverings commonly used in conventional heat therapy products.

Particular aspects of the disclosure can be implemented to realize one or more technical advantages over conventional heat patches. In some examples, the described heat patch provides targeted and sustained warmth to the postpartum abdominal area, thereby alleviating uterine cramps associated with uterine involution while promoting uterine recovery. In some examples, the heat patch improves localized blood circulation and facilitates the expulsion of lochia, thereby accelerating healing and reducing postpartum discomfort. In some examples, the multi-layer construction, garment-compatible adhesion, and contoured geometry collectively enable extended wear without significant displacement, garment damage, or user discomfort, improving usability during daily postpartum activities. As a result, aspects of the present disclosure provide a heat patch that is not merely a general warming product repurposed for postpartum use, but rather a system specifically configured to address the unique thermal, mechanical, and comfort-related demands of postpartum uterine recovery, while remaining simple to apply, non-medicated, and suitable for single-use deployment.

Although iron-based exothermic compositions are known for generating heat through oxidation, conventional heat-generating materials are typically selected and configured for direct skin application or short-duration use without regard to prolonged interaction with garments, adhesives, or sensitive postpartum conditions. In contrast, aspects of the present disclosure configure the heat-generating material to cooperate with surrounding layers to deliver a controlled and spatially distributed heat output suitable for garment-mediated postpartum use. The heat-generating material is selected and enclosed such that the rate and uniformity of heat generation are maintained within a therapeutic range while minimizing localized hot spots that could otherwise cause discomfort, adhesive degradation, or garment damage during extended wear.

In some aspects, the heat-generating material is encapsulated or distributed within a structure that regulates oxygen exposure to control the exothermic reaction rate, particularly in low-airflow environments created by clothing layers. This configuration enables consistent heat generation over extended durations without excessive temperature spikes or premature depletion of the heat-generating material. Such airflow-managed configurations differ from conventional heat patches that assume unrestricted ambient air exposure and do not account for reduced ventilation associated with garment-based postpartum use.

In some aspects, the heat-generating material functions as part of an integrated system in which the thermal output of the exothermic reaction is coordinated with the properties of the topsheet, intermediate layers, and attachment mechanism. By tailoring the interaction between the heat-generating material and the surrounding layers, the disclosed heat patch achieves a balance between effective uterine warming, user comfort, and mechanical compatibility with garments, rather than maximizing heat output alone as in conventional heat therapy products.

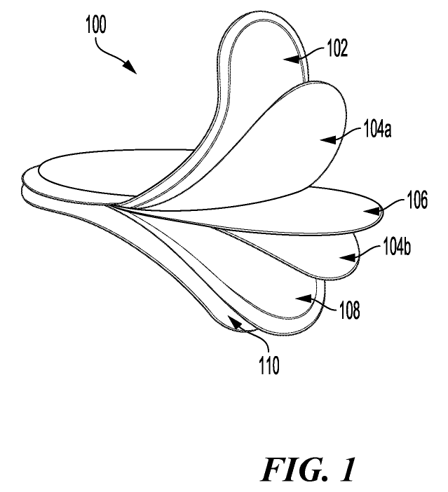

FIG. 1 is a diagram illustrating an example of a heat patch 100, in accordance with various aspects of the present disclosure. The heat patch 100 may also be referred to as a patch 100, hereinafter used interchangeably. As shown in the example of FIG. 1, the heat patch 100 may include a multi-layered structure configured to provide targeted and sustained warmth for pain relief, including postpartum uterine pain relief. A skin-facing layer of the heat patch 100 may include an ultra-plush soft topsheet 102, which may be formed from a non-woven material selected to be soft, breathable, and compatible with sensitive or recovering postpartum skin. The topsheet 102 may further provide a tactile buffer that moderates initial thermal sensation while permitting effective heat transfer from underlying layers. In some examples, the topsheet 102 may comprise a moisture-wicking material configured to draw perspiration away from the skin during extended wear, thereby improving comfort and reducing skin irritation.

Positioned beneath the topsheet 102 is a first polyethylene (PE) film layer 104a. The PE film layer 104a may function as a containment and distribution layer that separates the skin-facing topsheet 102 from an underlying heat-generating component 106, while promoting uniform lateral heat distribution across the heat patch 100. The heat-generating component 106 may comprise an exothermic chemical mixture configured to generate heat upon exposure to oxygen. In some examples, the mixture includes iron powder (about 30–40% by weight), activated carbon (about 10–20% by weight), vermiculite (about 20–30% by weight), sodium chloride (about 5–10% by weight), and sodium polyacrylate (about 10–15% by weight), the mixture being selected to provide a controlled oxidation rate and sustained heat output suitable for extended, garment-mediated postpartum use. The heat-generating component 106 undergoes an oxidation reaction when exposed to air, thereby generating sustained heat over an extended duration, such as six to eight hours or more.

In some aspects, oxygen from ambient air reaches the heat-generating component 106 through a controlled diffusion path defined by one or more layers of the heat patch 100. Specifically, ambient air may first pass through the non-woven back sheet 108, which is gas permeable while supporting the adhesive layer, and then diffuse through the second PE film layer 104b. The second PE film layer 104b may be at least partially gas permeable, or include diffusion-permissive regions, such that oxygen can pass therethrough without permitting migration of the heat-generating material.

After passing through the second PE film layer 104b, oxygen enters a semi-permeable membrane retaining the heat-generating component 106. The semi-permeable membrane permits oxygen molecules to diffuse into the heat-generating component 106 at a controlled rate while physically retaining solid and gel-forming constituents of the exothermic chemical mixture. Oxygen may further diffuse within the heat-generating component 106 in a lateral direction, enabling substantially uniform oxidation across the heat patch 100.

In some aspects, a portion of oxygen ingress may additionally occur through the skin-facing side of the heat patch 100, including diffusion through the topsheet 102 and the first PE film layer 104a, such that oxygen reaches the heat-generating component 106 from multiple directions. This multi-directional diffusion path promotes sustained and regulated heat generation while reducing localized hot spots and preventing excessive reaction rates. By routing oxygen through one or more diffusion-permeable layers and interfaces rather than through open apertures, the heat patch 100 maintains controlled oxygen exposure even when worn beneath clothing or in low-airflow environments, thereby sustaining the exothermic reaction over time without excessive temperature spikes or premature depletion of the heat-generating material.

As shown in the example of FIG. 1, a second PE film layer 104b may be positioned beneath the heat-generating component 106. The second PE film layer 104b may provide additional containment, structural support, and protection for the heat-generating component 106, thereby enhancing safety and reliability during use. Beneath the second PE film layer 104b, the heat patch 100 includes a non-woven back sheet 108 that forms a garment-facing surface of the heat patch 100. The back sheet 108 may include an adhesive layer configured to attach the heat patch 100 to a garment, such as postpartum underwear or similar apparel, during use. The adhesive may be selected to maintain attachment under sustained elevated temperatures generated by the heat-generating component 106 while avoiding excessive bonding, tearing, or deformation of delicate textile substrates upon removal.

As shown in the example of FIG. 1, to protect the adhesive prior to use, the heat patch 100 may further include a release paper layer 110 removably coupled to the back sheet 108. The release paper layer 110 may be peeled away by a user before application of the heat patch 100. In some examples, the adhesive associated with the back sheet 108 is formulated to be skin-safe and low-residue, and in some configurations may be suitable for optional direct skin application. For such configurations, the adhesive may be hypoallergenic and breathable to ensure compatibility with sensitive postpartum skin.

In some aspects, the heat patch 100 may include one or more additional layers or features, such as a moisture-wicking layer positioned between the topsheet 102 and the first PE film layer 104a to reduce perspiration during extended wear, or a temperature-regulating mechanism configured to maintain heat output from the heat-generating component 106 within a desired therapeutic range. The multi-layered construction and contoured configuration of the heat patch 100 provide advantages over conventional heat patches by enabling controlled, localized warmth, improved comfort, and reliable garment-based attachment, making the heat patch 100 particularly suitable for addressing postpartum uterine pain and discomfort.

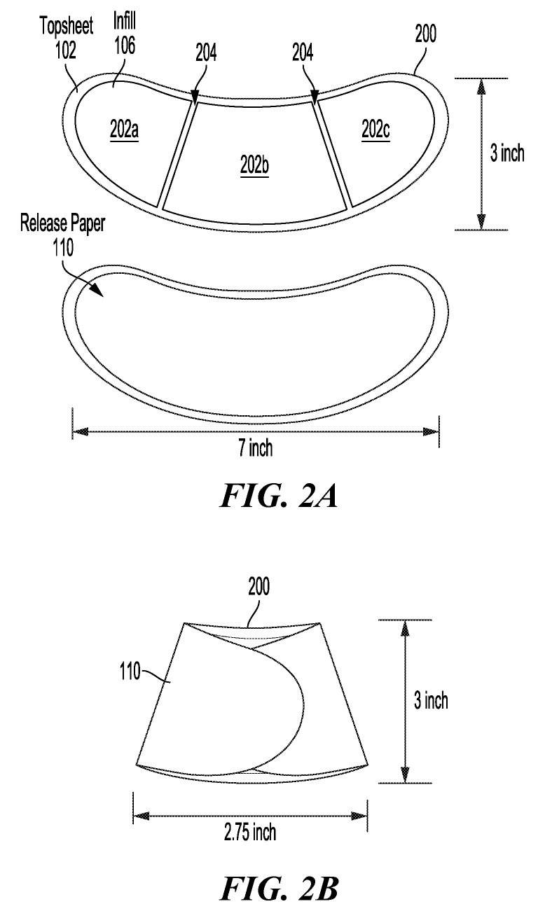

The heat patch 100 described with reference to FIG. 1 may take various forms, shapes, sizes, and configurations. By way of example and not limitation, the heat patch 100 may be implemented in any of the forms described with reference to FIGS. 2A, 2B, 3A, 3B, 4, 5, and 6, each of which illustrates an example aspect incorporating the multi-layer structural arrangement of the heat patch 100. Accordingly, the aspects shown in FIGS. 2A, 2B, 3A, 3B, 4, 5, and 6 represent illustrative variations of the heat patch 100 and do not limit the scope of the present disclosure.

FIG. 2A is a diagram illustrating an example of a heat patch 200, in accordance with various aspects of the present disclosure. As shown in the example of FIG. 2A, the heat patch 200 is formed with a generally curved, elongated profile configured to conform to the postpartum abdominal region when worn on or in association with a garment. The heat patch 200 is one example implementation of the heat patch 100 described with reference to FIG. 1, and includes the multi-layer structural layout of the heat patch 100, including the topsheet, heat-generating infill, intermediate film layers, adhesive backing, and release layer. In the example of FIG. 2, the curved geometry aligns with a natural contour of a lower abdomen, thereby improving surface contact and reducing localized gaps that could otherwise result in uneven heat delivery. In some examples, the heat patch 200 has an overall length of approximately 7 inches and a width of approximately 3 inches, although other dimensions are contemplated. As illustrated in the example of FIG. 2A, the heat patch 200 has a generally elongated, arcuate shape with opposing curved end portions and a concave upper edge and a convex lower edge. As discussed, the curvature of the heat patch 200 generally follows the contour of a lower abdominal region, such that the heat patch 200 extends laterally across the abdomen while maintaining a low profile when worn.

In some examples, the heat patch 200 includes multiple segmented panels 202a, 202b, and 202c that extend across the curved body of the heat patch 200. The segmented panels define flexible regions that allow the heat patch 200 to bend, flex, and adapt to movement of the wearer without significant wrinkling, bunching, or detachment. This segmentation further facilitates more uniform distribution of the heat-generating infill 106 across the surface area of the heat patch 200 by allowing the patch to maintain consistent proximity to the abdominal region during use.

Although the heat patch 200 is illustrated in FIG. 2A as including three segmented panels 202a, 202b, and 202c, aspects of the present disclosure are not limited to three segmented panels, and a greater or fewer number of panels may be employed. The number, size, and arrangement of segmented panels may be selected based on desired flexibility, coverage, and thermal distribution characteristics. In the illustrated example, the three-panel configuration enables the heat patch 200 to be folded into a compact form, as shown in FIG. 2B, while maintaining structural integrity of the heat-generating infill and allowing the heat patch 200 to unfold to its contoured shape during use.

The segmented panels 202a, 202b, and 202c may be separated by panel boundaries 204. The panel boundaries 204 define regions of increased flexibility relative to adjacent panel regions and may function as fold regions, hinge regions, or flex zones that allow the heat patch 200 to bend, flex, or fold in a controlled manner. In some aspects, the panel boundaries 204 facilitate folding of the heat patch 200 for compact storage or packaging, while also allowing the heat patch 200 to conform to the contours and movement of the postpartum abdomen during use.

The panel boundaries 204 may be formed by one or more of changes in material thickness, localized bonding patterns, scoring, pleating, or material transitions between adjacent layers, although the present disclosure is not limited to any particular implementation. By defining controlled fold regions without interrupting the continuity of the heat-generating infill layer, the panel boundaries 204 help maintain substantially uniform heat distribution across the heat patch 200 while reducing mechanical stress on the infill and adhesive layers during folding and wear.

The heat patch 200 comprises multiple layers, each contributing to thermal performance and user comfort. The infill layer 106 includes an iron-based exothermic formulation that generates heat upon controlled exposure to ambient air. In use, the infill layer 106 may provide sustained warmth within a therapeutic temperature range, such as approximately 40–50°C (e.g., 104-122°F), for several hours (for example, six to eight hours or more), although other temperature ranges and durations are contemplated.

Positioned above the infill layer is a plush topsheet 102 formed from a soft, breathable non-woven material. The topsheet 102 provides a skin-facing interface that enhances comfort during extended wear and moderates the perception of heat while allowing effective heat transfer. On the opposite side, the heat patch 200 includes a garment-facing back sheet with an adhesive layer covered by a removable release paper 110, which protects the adhesive prior to use and may be removed to attach the heat patch 200 to clothing.

The segmented construction of the heat patch 200 cooperates with the curved geometry to reduce mechanical stress on the infill layer and adhesive during movement, thereby helping maintain consistent heat application across the postpartum abdominal region. In some aspects, the heat patch 200 may be offered in alternative sizes, curvatures, or segment configurations to accommodate different body types or coverage preferences. In some examples, one or more layers of the heat patch 200 may incorporate moisture-wicking properties to reduce perspiration and improve comfort during prolonged wear, or stretch zones that provide additional adaptability while maintaining even thermal output.

Although the infill layer 106 is illustrated in FIG. 2A for purposes of clarity and explanation, the infill layer 106 is not exposed during use. As described with reference to FIG. 1, the infill layer 106 is enclosed (e.g., sandwiched) between one or more intermediate layers, including one or more polymer film layers and outer non-woven layers, such that the heat-generating material remains contained within the heat patch 200 during storage and use.

FIG. 2B illustrates the heat patch 200 in a folded configuration, in accordance with various aspects of the present disclosure. In the folded state, the heat patch 200 may have approximate dimensions of 2.75 inches in width and 3 inches in height, providing a compact form factor that facilitates storage, packaging, and transport. The folding arrangement is selected such that edges and segment boundaries overlap in a manner that reduces creasing or compression of the infill layer and preserves the integrity of the heat-generating material and adhesive. Upon unfolding, the heat patch 200 is configured to return substantially to its original contoured shape, thereby maintaining uniform heat distribution and adhesion performance during use. In some examples, the heat patch 200 may be individually packaged within a protective pouch to maintain hygiene and performance prior to application.

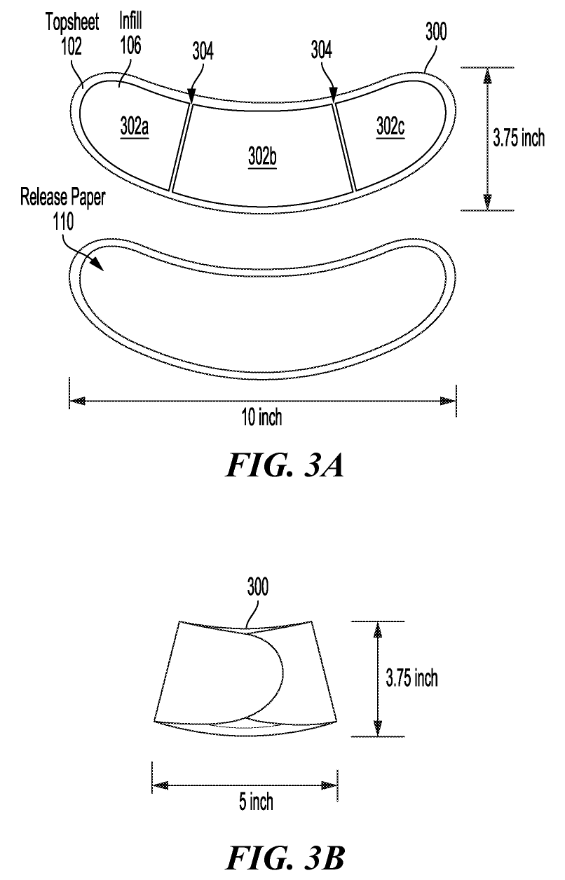

FIG. 3A is a diagram illustrating an example of a heat patch 300, in accordance with various aspects of the present disclosure. The heat patch 300 represents one example aspect of the heat patch 100 described with reference to FIG. 1, and may incorporate the same multi-layer structural layout, including a skin-facing topsheet, a heat-generating infill layer enclosed between intermediate layers, an adhesive-backed garment-facing layer, and a removable release layer. As shown in the example of FIG. 3A, the heat patch 300 has a generally elongated, arcuate profile with a larger curvature than the heat patch 200 described with reference to FIGS. 2A and 2B. The increased curvature is selected to follow the contour of a broader portion of the postpartum abdominal region.

In the example of FIG. 3A, the heat patch 300 has an overall length of approximately 10 inches and a width of approximately 3.75 inches, although these dimensions are provided by way of example and other sizes are contemplated. The non-rectangular, contoured shape of the heat patch 300 includes opposing curved end portions and a central body portion, allowing the heat patch 300 to extend laterally across the abdomen while maintaining a low-profile fit when worn. This configuration provides increased surface coverage for delivering heat to the uterine region and surrounding areas.

As illustrated in FIG. 3A, the heat patch 300 includes a removable release paper 110 disposed on a garment-facing side of the heat patch 300. The release paper 110 covers and protects an adhesive layer prior to use, preventing premature adhesion and contamination during storage and handling. The release paper 110 may be formed from a coated paper, polymer film, or other suitable release material configured to be peeled away by a user immediately before application of the heat patch 300 to a garment or other surface. Removal of the release paper 110 exposes the adhesive layer, thereby enabling the heat patch 300 to be secured in place during use.

The heat patch 300 further includes segmented panels 302a, 302b, and 302c, which are separated by panel boundaries 304. The panel boundaries 304 define regions of increased flexibility relative to adjacent panel regions and may function as fold regions, hinge regions, or flex zones that allow the heat patch 300 to bend, flex, or fold in a controlled manner. In some aspects, the panel boundaries 304 facilitate folding of the heat patch 300 for compact storage or packaging, while also allowing the heat patch 300 to conform to the contours and movement of the postpartum abdomen during use.

The panel boundaries 304 may be formed by one or more of changes in material thickness, localized bonding patterns, scoring, pleating, or material transitions between adjacent layers, although the present disclosure is not limited to any particular implementation. By defining controlled fold regions that accommodate bending of the heat patch 300 while maintaining operational continuity of the heat-generating infill layer across adjacent panels, the panel boundaries 304 may help promote substantially uniform heat distribution across the heat patch 300 and reduce mechanical stress on the infill and adhesive layers during folding and wear.

In some examples, the panel boundaries 304 may be formed by one or more of changes in material thickness, localized bonding patterns, scoring, pleating, or material transitions between adjacent layers, although the present disclosure is not limited to any particular implementation. In some aspects, the controlled fold regions defined by the panel boundaries 304 may at least partially interrupt the heat-generating infill layer (for example, by reducing or omitting infill material within the fold region), while in other aspects the heat-generating infill layer may remain continuous across the fold regions. In either aspect, as discussed, the panel boundaries 304 may be configured to accommodate bending of the heat patch 300 while maintaining operational continuity of the heating function across adjacent panels so as to help promote substantially uniform heat distribution across the heat patch 300.

In various aspects, the heat patch 300 includes a soft, non-woven topsheet 102 configured to be gentle on sensitive postpartum skin while maintaining breathability and comfort during extended wear. Beneath the topsheet 102, a heat-generating infill layer 106 may include an iron-based exothermic formulation provides sustained warmth within a therapeutic temperature range, such as approximately 40–50°C, for several hours, although other temperature ranges and durations are contemplated. A polyethylene (PE) backing layer and adhesive associated therewith enable the heat patch 300 to be removably attached to clothing, such as postpartum underwear, providing convenience and stability during use. In the example of FIG. 3A, the infill layer 106 is not exposed during use. As described with reference to FIG. 1, the infill layer 106 is enclosed and sandwiched between one or more intermediate layers, including one or more polymer film layers and outer non-woven layers, such that the heat-generating material remains contained within the heat patch 300 during storage and use.

FIG. 3B illustrates an example of the heat patch 300 in a folded configuration, in accordance with various aspects of the present disclosure. In the folded state, the heat patch 300 may have approximate dimensions of 5 inches in width and 3.75 inches in height, thereby providing a compact form factor suitable for storage, packaging, and transport. The folding arrangement may be defined by panel boundaries 304 (shown in FIG. 3A) or fold regions such that the heat-generating infill and adhesive layers are not excessively creased or compressed. Upon unfolding, the heat patch 300 is configured to substantially return to its original contoured shape, thereby maintaining uniform heat distribution and reliable attachment during application.

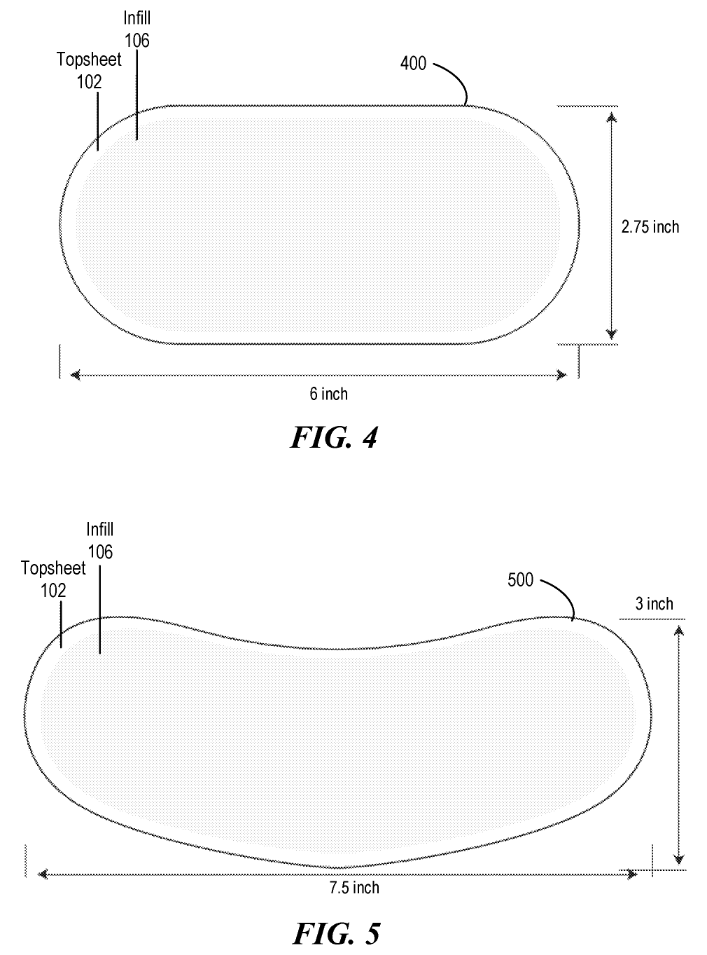

FIG. 4 is a diagram illustrating an example of a heat patch 400, in accordance with various aspects of the present disclosure. The heat patch 400 represents another example of the heat patch 100 described with reference to FIG. 1 and incorporates the same multi-layer structural layout, including a skin-facing topsheet 102, a heat-generating infill layer 106 enclosed between one or more intermediate layers, a garment-facing layer with adhesive, and a removable release layer. As shown in FIG. 4, the heat patch 400 has a generally elongated, pill-shaped or capsule-shaped profile defined by rounded end portions and substantially straight lateral edges. In the example of FIG. 4, the heat patch 400 has approximate dimensions of 6 inches in length and 2.75 inches in width, although these dimensions are provided by way of example, and other sizes and proportions are contemplated.

The pill-shaped geometry of the heat patch 400 may be particularly suited for providing targeted heat therapy to localized regions of the body, such as the postpartum abdominal area, where focused warming is desired rather than broad lateral coverage. The rounded perimeter reduces edge concentration and pressure points, thereby improving comfort during wear and helping maintain uniform contact with the underlying surface. The shape further facilitates even lateral heat distribution across the contact area, reducing the likelihood of localized hot spots during use.

The heat patch 400 may be constructed with a multi-layer configuration consistent with other aspects described herein. The core heat-generating component comprises an iron-based exothermic formulation that generates heat upon controlled exposure to ambient air. In use, the heat patch 400 may deliver sustained and controlled thermal output within a therapeutic temperature range, such as approximately 40–50°C, for several hours, although other temperature ranges and durations are contemplated. A skin-facing topsheet formed from a soft, breathable non-woven material provides comfort during extended wear while permitting effective heat transfer from the infill layer.

Although the heat patch 400 is illustrated as a unitary, non-segmented body, aspects of the present disclosure are not limited to the absence of segmentation. In some examples, the heat patch 400 may include one or more flexible regions, stretch zones, or panel boundaries to increase adaptability to body contours while maintaining the pill-shaped outer profile. Additionally, while the heat patch 400 is shown as having a generally symmetrical shape, asymmetric or tapered variations of the pill-shaped geometry may be employed to accommodate different anatomical regions or user preferences.

As with other aspects described herein, the heat patch 400 may be configured to be attached to clothing or, in some configurations, directly to the skin using an adhesive layer covered by a removable release liner prior to use. The heat patch 400 may be worn alone or in combination with garments and may be packaged individually or in sets. Accordingly, the pill-shaped heat patch 400 provides a compact, versatile alternative to larger contoured aspects, while retaining the same underlying thermal and structural principles described with reference to FIGS. 1, 2A, 2B, 3A, and 3B.

FIG. 5 is a diagram illustrating an example of a heat patch 500, in accordance with various aspects of the present disclosure. The heat patch 500 represents another example of the heat patch 100 described with reference to FIG. 1 and incorporates the same multi-layer structural layout, including a skin-facing topsheet 102, a heat-generating infill layer 106 enclosed between one or more intermediate layers, a garment-facing adhesive layer, and a removable release layer. As shown in FIG. 5, the heat patch 500 has a generally elongated, asymmetric curved profile, which may also be referred to as a tilted or offset curvature. In the example of FIG. 5, the heat patch 500 has an approximate length of 7.5 inches and a width of approximately 3 inches, although these dimensions are provided by way of example and other sizes and proportions are contemplated.

In the example of FIG. 5, the asymmetric curved geometry of the heat patch 500 is configured to follow the natural contours of the postpartum abdominal region, including variations in curvature across different portions of the abdomen. Unlike symmetric or uniformly curved shapes, the tilted configuration enables the heat patch 500 to provide more focused or preferential heat distribution along selected areas of the abdomen while still maintaining coverage across the uterine region. This configuration may be particularly advantageous for users experiencing localized discomfort or asymmetrical sensitivity during postpartum recovery.

In the example of FIG. 5, the heat patch 500 includes a multi-layer construction consistent with other aspects described herein. The core heat-generating component comprises an iron-based exothermic formulation that generates heat upon controlled exposure to ambient air. In use, the heat patch 500 may deliver sustained thermal output within a therapeutic temperature range, such as approximately 40–50°C, for several hours, although other temperature ranges and durations are contemplated. A skin-facing topsheet formed from a soft, breathable non-woven material provides comfort during extended wear while permitting effective heat transfer from the underlying infill layer.

In some aspects, the heat patch 500 may be formed as a unitary body without segmented panels. In other aspects, one or more flexible regions, stretch zones, or panel boundaries may be incorporated into the heat patch 500 to further enhance adaptability to body movement while preserving the overall tilted curved profile. Additionally, while the illustrated aspect shows a particular direction and degree of curvature, aspects of the present disclosure are not limited to that configuration, and mirrored, inverted, or otherwise modified asymmetric curvatures may be employed to accommodate different anatomical orientations or user preferences.

As with other aspects described herein, the heat patch 500 may be configured to be removably attached to clothing, such as postpartum underwear, using an adhesive layer protected by a release liner prior to use. In some configurations, the heat patch 500 may alternatively be adapted for direct skin application. Accordingly, the tilted and asymmetrically curved heat patch 500 provides an additional geometric option within the disclosed heat patch system, enabling tailored heat delivery while retaining the same underlying thermal and structural principles described with reference to FIGS. 1, 2A, 2B, 3A, and 3B.

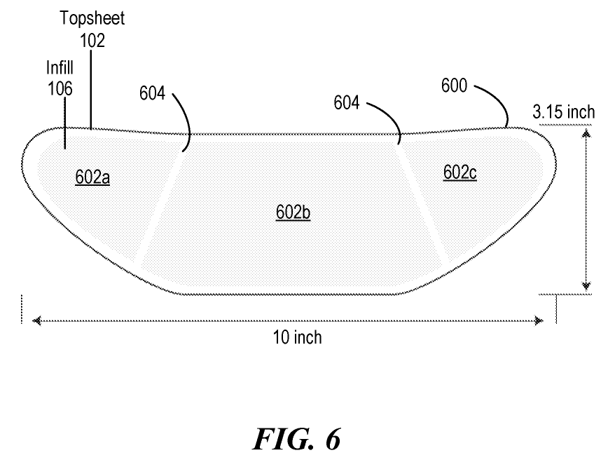

FIG. 6 is a diagram illustrating an example of a heat patch 600, in accordance with various aspects of the present disclosure. The heat patch 600 represents another example of the heat patch 100 described with reference to FIG. 1 and incorporates the same multi-layer structural layout, including a skin-facing topsheet 102, a heat-generating infill layer 106 enclosed between intermediate layers, a garment-facing adhesive layer, and a removable release layer. As shown in FIG. 6, the heat patch 600 has a generally trapezoidal profile with a wider upper edge and a narrower lower edge, defining an ergonomic shape selected to correspond to the contour of the lower abdominal region.

In the example of FIG. 6, the heat patch 600 has an overall length of approximately 10 inches and a width of approximately 3.15 inches, although these dimensions are provided by way of example and other sizes, proportions, and aspect ratios are contemplated. The trapezoidal geometry enables broader lateral coverage across the abdomen while maintaining a reduced profile toward the lower edge, which may improve comfort, reduce bunching, and promote uniform contact during movement. This shape may be suitable for users seeking coverage across a larger abdominal area while maintaining a stable fit.

The heat patch 600 may include multiple segmented panels 602a, 602b, and 602c, which may be separated by panel boundaries 604. The panel boundaries 604 define regions of increased flexibility relative to adjacent panel regions and may function as fold regions, hinge regions, or flex zones that allow the heat patch 600 to bend and conform to the natural curvature and movement of the body. In some aspects, the panel boundaries 604 distribute mechanical strain across the heat patch 600, thereby reducing stress on the heat-generating infill and adhesive layers during wear.

The segmented panel configuration of the heat patch 600 promotes substantially uniform heat distribution across the trapezoidal body while allowing the heat patch 600 to adapt to anatomical contours without compromising structural integrity. Although three segmented panels are illustrated, aspects of the present disclosure are not limited to this configuration, and a greater or fewer number of panels, or alternative flexible regions, may be employed depending on desired flexibility, coverage, or folding characteristics.

The construction of the heat patch 600 includes a soft, breathable non-woven topsheet configured to be gentle on sensitive postpartum skin while permitting effective heat transfer from the underlying infill layer. Beneath the topsheet, the heat-generating component comprises an iron-based exothermic formulation that generates sustained heat through a controlled oxidation reaction when exposed to ambient air. In use, the heat patch 600 may deliver therapeutic warmth within a temperature range such as approximately 40–50°C for several hours, although other temperature ranges and durations are contemplated.

As with other aspects described herein, the heat patch 600 may be removably attached to clothing, such as postpartum underwear, using an adhesive layer protected by a release liner prior to use. In some configurations, the heat patch 600 may alternatively be adapted for direct skin application. Accordingly, the trapezoidal heat patch 600 provides an additional geometric embodiment within the disclosed heat patch system, offering expanded coverage and flexibility while retaining the same underlying thermal and structural principles described with reference to FIGS. 1, 2A, 2B, 3A, and 3B.

In some aspects, the heat patch 600 may be configured to be folded for compact storage or packaging in a manner similar to the folded configurations described with reference to FIGS. 2B and 3B. For example, the panel boundaries 604 may define fold regions that allow the heat patch 600 to be folded without substantially creasing or compressing the heat-generating infill layer. Upon unfolding, the heat patch 600 may substantially return to its original trapezoidal shape, thereby maintaining uniform heat distribution and attachment performance during use.

As discussed herein, various aspects of the present disclosure address the challenge of postpartum uterine pain and discomfort by providing a single-use heat patch specifically configured for uterine recovery. The heat patch may be removably attached to the inside of clothing, such as postpartum underwear, and is configured to activate upon exposure to ambient air to deliver therapeutic warmth toward the abdominal region for an extended duration. Unlike conventional heat patches intended for general pain relief, the disclosed heat patch is designed with size, shape, and attachment characteristics tailored to the postpartum body, enabling precise and consistent heat delivery to one or more targeted uterine regions while maintaining comfort during movement and extended wear.

By delivering sustained warmth to the postpartum abdominal area, the heat patch helps soothe muscles, reduce tension, and alleviate postpartum uterine cramps. The therapeutic heat further promotes uterine recovery by improving localized blood circulation, which supports uterine contraction and healing following childbirth, including healing associated with the significant internal wound created by placental detachment. Improved circulation may also facilitate expulsion of lochia, reduce swelling, and accelerate the overall recovery process, thereby improving comfort and mobility for new mothers during the postpartum period.

In some examples, the heat patch may deliver sustained warmth within a therapeutic temperature range, such as approximately 40–50°C, for an extended duration, which may range from several hours to up to approximately 20 hours, depending on formulation and configuration. In some aspects, the heat patch reaches its therapeutic temperature within approximately 15–30 minutes of activation. The adhesive associated with the heat patch maintains secure attachment to clothing under sustained elevated temperatures while avoiding excessive adhesion that could tear or damage delicate garment materials upon removal. This balance enables reliable placement during use while preserving garment integrity, which is particularly advantageous when used with disposable or lightweight postpartum underwear.

The heat patch may be formed in a variety of non-limiting shapes and configurations, including aspects having medium curvature, larger curvature, trapezoidal profiles, pill-shaped profiles, or asymmetric curved geometries, as described with reference to FIGS. 2A, 2B, 3A, 3B, 4, 5, and 6. These shapes are selected to conform to different anatomical contours of the postpartum abdomen and to accommodate user preferences for broader coverage or more localized heat delivery. In some aspects, segmented paneling is employed to enhance flexibility and adaptability, allowing the heat patch to conform to natural body movement without undesirable folding, creasing, or displacement during use.

In some examples, the heat patch incorporates moisture-wicking materials to reduce perspiration and improve comfort during prolonged wear. In some configurations, the adhesive formulation may be hypoallergenic and breathable, allowing optional direct skin application in addition to garment-based attachment. Variations in size, shape, curvature, panel configuration, heat duration, and adhesive strength may be employed to provide different coverage profiles or comfort characteristics. Accordingly, the disclosed heat patch system provides a versatile platform that can be tailored to individual postpartum recovery needs while retaining the underlying structural and thermal principles described herein.

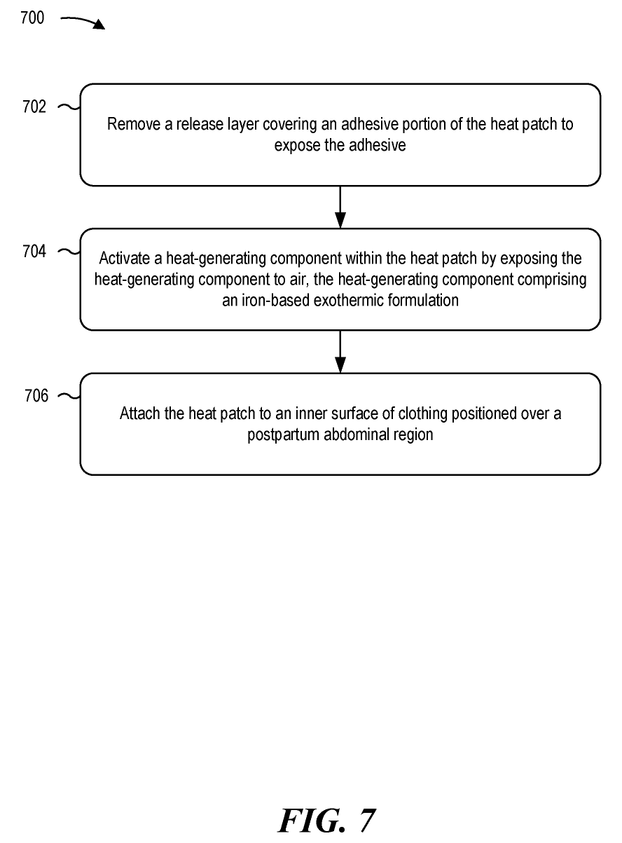

FIG. 7 is a flow diagram illustrating an example of a process 700 for using a heat patch in accordance with various aspects of the present disclosure. The heat patch may be an example of a heat patch 100, 200, 300, 400, 500, or 600 described with reference to FIGS. 1, 2A, 2B, 3A, 3B, 4, 5, and 6, respectively. The process 700 may be performed by a user or by another person assisting the user. As shown in the example of FIG. 7, the process 700 begins at block 702 by removing a release layer covering an adhesive portion of the heat patch to expose the adhesive. In some examples, the process 700 further includes unfolding the heat patch from a folded configuration, such as from a folded state configured for storage or packaging, prior to or after removal of the release layer, thereby allowing the heat patch to assume a contoured shape for application.

In some examples, removal of the release layer initiates exposure of a heat-generating component to ambient air, thereby activating the heat-generating component, which comprises an iron-based exothermic formulation. The heat-generating component is enclosed within the heat patch between a breathable, non-woven topsheet forming a skin-facing surface and a non-woven backing layer comprising the adhesive, with at least a first polyethylene (PE) film layer positioned between the topsheet and the heat-generating component and, in some aspects, a second PE film layer positioned between the heat-generating component and the backing layer. At block 704, the process 700 includes activating a a heat-generating component within the heat patch by exposing the heat-generating component to air, the heat-generating component comprising an iron-based exothermic formulation. At block 706, the process 700 includes attaching the heat patch to an inner surface of clothing positioned over a postpartum abdominal region, such as postpartum underwear, using the exposed adhesive. In such examples, attaching the heat patch results in the heat patch delivering heat to the postpartum abdominal region for a sustained period.

In some examples, the heat patch is positioned and aligned with a postpartum uterine area prior to or during attachment such that the heat patch delivers heat to the postpartum abdominal region for a sustained period. The heat patch may include a plurality of segmented panels, and in some aspects each segmented panel includes a pouch containing a portion of the heat-generating component, enabling flexibility and conformability while maintaining uniform heat delivery. During use, the heat patch may maintain a therapeutic temperature range of approximately 40–50°C and may provide localized warmth for up to approximately 20 hours. The delivered heat may be used to alleviate postpartum uterine cramps, including afterpains associated with oxytocin-induced uterine contractions, improve blood circulation, and support uterine recovery. After completion of the heating period, the heat patch may be removed from the clothing and disposed of, and the process 700 may be repeated using a new heat patch as needed.

In some examples, the heat patch used in process 700 may have a curved shape, a trapezoidal shape, or a pill-shaped profile, including but not limited to shapes having dimensions of approximately 7.5 inches by 3 inches, 10 inches by 3.15 inches, or 6 inches by 2.75 inches, respectively, to provide broader or more localized heat coverage depending on user preference and anatomical considerations.

In some examples, a heat patch may be manufactured using a laminated construction process. For example, a heat-generating component configured to produce sustained heat through an exothermic chemical reaction, such as an iron-based composition, may be provided in bulk form or as individual pouches. A breathable non-woven topsheet may be supplied and laminated to a first polyethylene (PE) film layer so that the first PE film layer is positioned between the non-woven topsheet and the heat-generating component in the finished heat patch. The heat-generating component may then be disposed adjacent to the first PE film layer on a side of the first PE film layer opposite the non-woven topsheet.

A non-woven backing layer including an adhesive suitable for attachment to an inside surface of an article of clothing may be provided and joined to the heat-generating component so that the adhesive is positioned on a side of the non-woven backing layer opposite the heat-generating component. A release layer may be applied over the exposed adhesive on the non-woven backing layer to protect the adhesive prior to use. The resulting laminated structure, which may include the topsheet, the first PE film layer, the heat-generating component, a second PE film layer positioned between the heat-generating component and the non-woven backing layer, the non-woven backing layer, and the release layer, may then be cut or otherwise formed to define a contoured shape configured to fit an abdominal region.

In some implementations, the laminated structure may be cut or sealed to form a group of segmented panels. Each panel may include a respective pouch, and each pouch may contain a portion of the heat-generating component. The segmented panels may facilitate bending or folding of the heat patch while maintaining desired heat distribution across the abdominal region. The cutting or forming operations may also be used to generate specific geometries, such as a first curved heat patch having dimensions of approximately 7.5 inches by 3 inches, a second curved heat patch having dimensions of approximately 10 inches by 3.75 inches, a trapezoid having dimensions of approximately 10 inches by 3.15 inches, or a pill-shape having dimensions of approximately 6 inches by 2.75 inches, although other shapes and dimensions may be used.

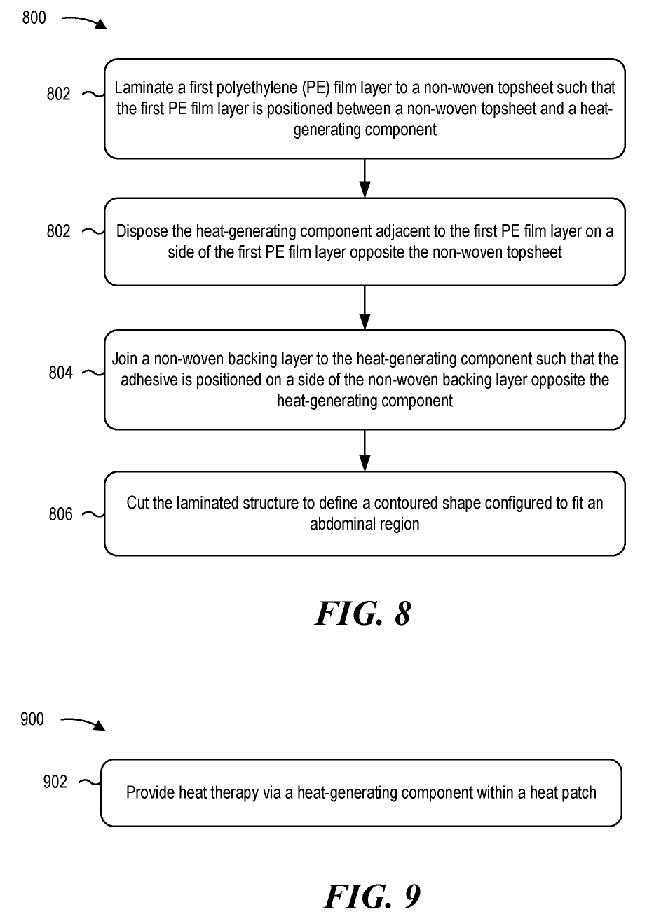

FIG. 8 illustrates an example process 800 for manufacturing a heat patch, in accordance with various aspects of the present disclosure. Although FIG. 8 depicts operations in a particular order for purposes of illustration, the ordering of the operations is not limiting, and one or more operations may be omitted, combined, performed in parallel, or performed in a different order. In some examples, the heat patch manufactured in the process 800 may be an example of the heat patch 100, 200, 300, 400, 500, or 600 described with reference to FIGS. 1, 2A, 2B, 3A, 3B, 4, 5, and 6, respectively.

The process 800 may include providing a heat-generating component and a breathable non-woven topsheet. In some examples, the heat-generating component includes an iron-based composition configured to undergo an exothermic chemical reaction in the presence of oxygen, moisture, or both to produce sustained heat over a target time interval. The heat-generating component may be supplied as a bulk mixture, as discrete metered portions, or pre-packaged in one or more pouches. The non-woven topsheet may be formed from one or more synthetic or natural fibers and may be configured to form a skin-facing surface of the finished heat patch. The topsheet may be selected to provide a desired combination of softness, breathability, and mechanical strength.

At block 802, a first polyethylene (PE) film layer is laminated to the non-woven topsheet. In some examples, thermal bonding, adhesive bonding, ultrasonic bonding, or other lamination techniques may be used. The lamination process positions the first PE film layer between the non-woven topsheet and the heat-generating component in the final assembly and may be configured to control vapor transmission, air permeability, and mechanical integrity.

At block 804, a heat-generating component is disposed adjacent to the first PE film layer on a side of the first PE film layer opposite the non-woven topsheet. For example, the heat-generating component may be placed or deposited into one or more cavities, pockets, or pouches defined by or between layers of film and non-woven material. In some embodiments, the heat-generating component is distributed into a group of segmented regions corresponding to individual panels of the heat patch. The heat-generating component may comprise an iron-based composition.

In some examples, a non-woven backing layer comprising an adhesive is provided. The non-woven backing layer may include a pressure-sensitive adhesive or other attachment mechanism configured to secure the heat patch to an inside surface of an article of clothing. The backing layer may optionally be pre-laminated to a second PE film layer that is positioned between the heat-generating component and the backing layer in the finished product.

At block 806, a non-woven backing layer is joined to the heat-generating component. The joining may be performed by lamination, adhesive bonding, thermal bonding, ultrasonic bonding, or combinations thereof, such that the adhesive on the backing layer is positioned on a side of the backing layer opposite the heat-generating component. In some embodiments, the joining step at block 806 also defines or reinforces panel boundaries, pouches, or other internal structures of the heat patch.

In some implementations, a release layer may be applied over the exposed adhesive on the non-woven backing layer. The release layer may include a silicone-coated paper, a polymeric film, or other suitable liner configured to protect the adhesive during storage and transport and to be removed by the user prior to application of the heat patch.

At block 808, the resulting laminated structure is cut or otherwise formed to define an outer contour of the heat patch. The cutting or forming at block 808 may be configured to generate one or more predetermined shapes sized and contoured to fit an abdominal region or other anatomical region of interest. Example shapes may include curved profiles, trapezoidal profiles, pill-shaped profiles, or other geometries having specified lengths, widths, and curvature parameters. In some examples, the cutting at block 808 may also form a group of segmented panels, with each panel comprising a respective pouch containing at least a portion of the heat-generating component.

Process 800 may further include additional blocks, such as quality control inspection, packaging, application of labeling or instructions, and storage. One or more of the parameters associated with blocks 802–806, including material selections, layer thicknesses, adhesive properties, panel geometries, and cutting patterns, may be adjusted to tailor the thermal performance, flexibility, and fit characteristics of the heat patch for different user populations or use cases.

FIG. 9 illustrates an example process 900 for heat therapy, in accordance with various aspects of the present disclosure. Although FIG. 9 depicts operations in a particular order for purposes of illustration, the ordering of the operations is not limiting, and one or more operations may be omitted, combined, performed in parallel, or performed in a different order. As shown in the example of FIG. 9, the process 900 begins at block 902 by providing heat therapy via a heat-generating component within a heat patch. The heat-generating component may include an iron-based exothermic formulation configured to generate sustained heat upon controlled exposure to ambient air. In some examples, providing heat therapy includes activating the heat-generating component by exposing the heat patch to air, such as by removing a release layer from an adhesive-bearing backing layer and positioning the heat patch for use. The heat patch may include a non-woven topsheet forming a skin-facing surface, the topsheet being breathable, a first polyethylene (PE) film layer positioned between the non-woven topsheet and the heat-generating component, a non-woven backing layer comprising an adhesive layer for attaching the heat patch to the inside of clothing, and a release layer covering the adhesive prior to application. In some aspects, the heat patch is configured to achieve and maintain a therapeutic temperature range, such as approximately 40–50°C, for a sustained period during which the process 900 delivers localized warmth to a target body region, for example a postpartum abdominal region associated with uterine recovery.

As used herein, the term “determining” encompasses a wide variety of actions. For example, “determining” may include calculating, computing, processing, deriving, investigating, looking up (e.g., looking up in a table, a database, or another data structure), ascertaining and the like. Additionally, “determining” may include receiving (e.g., receiving information), accessing (e.g., accessing data in a memory) and the like. Furthermore, “determining” may include resolving, selecting, choosing, establishing, and the like.

As used herein, a phrase referring to “at least one of” a list of items refers to any combination of those items, including single members. As an example, “at least one of: a, b, or c” is intended to cover: a, b, c, a-b, a-c, b-c, and a-b-c.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

It is to be understood that the claims are not limited to the precise configuration and components illustrated above. Various modifications, changes, and variations may be made in the arrangement, operation, and details of the methods and apparatus described above without departing from the scope of the claims.

What is claimed is:

1. A heat patch, comprising:
a heat-generating component configured to produce sustained heat through an exothermic chemical reaction, the heat-generating component comprising an iron-based composition;
a non-woven topsheet forming a skin-facing surface, the topsheet being breathable;
a first polyethylene (PE) film layer positioned between the topsheet and the heat-generating component;
a non-woven backing layer comprising an adhesive for attaching the heat patch to the inside of clothing; and
a release layer covering the adhesive, the heat patch being contoured to fit an abdominal region.

2. The heat patch of claim 1, wherein the heat-generating component comprises:
iron powder in a range of 30-40% by weight,
activated carbon in a range of 10-20% by weight,
vermiculite in a range of 20-30% by weight,
sodium chloride in a range of 5-10% by weight, and
sodium polyacrylate in a range of 10-15% by weight.

3. The heat patch of claim 1, further comprising a second PE film layer positioned between the heat-generating component and the non-woven backing layer.

4. The heat patch of claim 1, wherein the non-woven topsheet comprises a moisture-wicking material.

5. The heat patch of claim 1, wherein the non-woven backing layer is configured to adhere securely to postpartum underwear.

6. The heat patch of claim 1, wherein:
the heat patch comprises a group of segmented panels;
each panel of the group of segmented panels comprises a pouch; and
each pouch includes the heat-generating component.

7. The heat patch of claim 1, wherein a shape of the heat patch is one of:
a first curved heat patch having dimensions of approximately 7.5 inches by 3 inches;
a second curved heat patch having dimensions of approximately 10 inches by 3.75 inches;
a trapezoid having dimensions of approximately 10 inches by 3.15 inches; or
a pill-shape having dimensions of approximately 6 inches by 2.75 inches.

8. The heat patch of claim 1, wherein the heat-generating component is encapsulated in a semi-permeable membrane to allow air exposure.

9. The heat patch of claim 1, wherein the heat patch provides a therapeutic temperature range of 40-50°C.

10. The heat patch of claim 1, wherein the heat patch is a single use heat patch.

11. A method of manufacturing a heat patch, the method comprising:
laminating a first polyethylene (PE) film layer to a non-woven topsheet;
disposing a heat-generating component adjacent to the first PE film layer on a side of the first PE film layer opposite the non-woven topsheet, the heat-generating component comprising an iron-based composition;
joining a non-woven backing layer to the heat-generating component, the non-woven backing layer comprising an adhesive configured for attaching the heat patch to an inside surface of an article of clothing, the adhesive being positioned on a side of the non-woven backing layer opposite the heat-generating component; and
cutting the laminated structure to define a contoured shape configured to fit an abdominal region.

12. The method of claim 11, wherein the heat patch comprises a group of segmented panels.

13. The method of claim 12, wherein:
each panel of the group of segmented panels includes a pouch; and
each pouch includes the heat-generating component.

14. The method of claim 11, wherein a shape of the heat patch is one of:
a first curved heat patch having dimensions of approximately 7.5 inches by 3 inches;
a second curved heat patch having dimensions of approximately 10 inches by 3.75 inches;
a trapezoid having dimensions of approximately 10 inches by 3.15 inches; or
a pill-shape having dimensions of approximately 6 inches by 2.75 inches.

15. A method for heat therapy, the method comprising:
providing heat therapy via a heat-generating component within a heat patch, the heat-generating component comprising an iron-based exothermic formulation, the heat patch comprising:
a non-woven topsheet forming a skin-facing surface, the topsheet being breathable;
a first polyethylene (PE) film layer positioned between the non-woven topsheet and the heat-generating component;
a non-woven backing layer comprising an adhesive layer for attaching the heat patch to the inside of clothing; and
a release layer covering the adhesive prior to application.

16. The method of claim 15, further comprising: attaching the heat patch to an inner surface of clothing positioned over a postpartum abdominal region, such that the heat patch delivers heat to the postpartum abdominal region.

17. The method of claim 15, wherein the heat patch is attached to postpartum underwear via the adhesive layer.

18. The method of claim 15, further comprising applying the heat patch to alleviate afterpains caused by oxytocin-induced uterine contractions.

19. The method of claim 15, wherein the heat patch further comprises a second PE film layer positioned between the heat-generating component and the non-woven backing layer.

20. The method of claim 15, wherein a shape of the heat patch is one of:
a first curved heat patch having dimensions of approximately 7.5 inches by 3 inches;
a second curved heat patch having dimensions of approximately 10 inches by 3.75 inches;
a trapezoid having dimensions of approximately 10 inches by 3.15 inches; or
a pill-shape having dimensions of approximately 6 inches by 2.75 inches.
